(19) United States
(12) Patent Application Publication
(10) Pub. No.: US 2015/0238651 A1
(43) Pub. Date: Aug. 27, 2015

(54) Pressure Sensitive Adhesive Compositions Containing Plant and Animal Butters

(71) Applicant: Euromed, Inc,, Orangeburg, NY (US)

(72) Inventors: Ying Dong, Pomona, NY (US); Ravi Ramjit, Dix Hills, NY (US); Shridhar Gangolli, Park Ridge, NJ (US)

(21) Appl. No.: 14/424,472

(22) PCT Filed: Aug. 29, 2013
(86) PCT No.: PCT/US13/57239

§ 371 Date: Feb. 27, 2015

Related U.S. Application Data
(63) Continuation of application No. 13/971,384, filed on Aug. 20, 2013, now abandoned.
(60) Provisional application No. 61/694,738, filed on Aug. 29, 2012.

(51) Int. Cl. A61L 24/00; A61L 24/06

(52) U.S. Cl. CPC A61L 24/0005; A61L 24/06; A61L 2400/14

(57) ABSTRACT

The present invention relates to an adhesive composition applicable to skin comprising a shea butter and /or at least one homopolymer, and/or copolymer. This invention also relates to a medical adhesive device and cosmetic adhesive applications including such adhesive composition.

### CROSS REFERENCE TO RELATED APPLICATIONS

This application claims the benefit of U.S. Ser. No. 61/694,738 filed on Aug. 29, 2012.

### FIELD OF THE INVENTION

The present invention relates to a pressure sensitive, moldable adhesive dressing composition suitable for various medical device applications, particularly ostomy care applications to prevent leakage around the stoma which can lead to damage of healthy neighboring skin. Furthermore, the adhesive dressing composition can be molded to maximize coverage and protection of various abdominal anatomies, sealing the area between the skin and ostomy appliance. In particular, the present invention relates to a pressure sensitive adhesive composition comprising polyethylene copolymers, polystyrene copolymers, poly-isobutylene copolymers and various butters including, but not limited to, shea butter, avocado butter, cocoa butter, aloe butter and animal butters such as butter derived from bovine milk.

### BACKGROUND OF THE INVENTION

Pressure sensitive adhesives for ostomy appliances and wound dressing have been used for many years. For example, hydrocolloid adhesives containing hydrophilic absorbents, which absorb fluids and moisture, are one type of unique pressure sensitive adhesives in medical devices. Patients with surgery for a variety of diseases related to the ostomy or urinary tract, typically cancer, have to wear the collecting device permanently. Thus, adhesion of a collecting device to body skin must securely stay in place and have enough ability to absorb the fluids and moisture without detaching. When removing the adhesive, the adhesive should be cohesive enough to remove without leaving residues on the skin, while at same time avoiding damage to the skin.

Conventional pressure sensitive adhesives embody these functions, such as adhering to skin and providing a barrier to leakage. There are, however, few products which consider skincare, skin protection, or even promoting skin health.

Thus, there is a need for pressure sensitive adhesives with skin care properties in mind. Specifically, developing adhesives that have the properties to protect and prevent skin disease or even provide skincare functions while still having a good moisture absorption and fixation is particularly important when a medical device has to be worn on the skin for a long time to protect the skin from corrosive fluids discharged by the stoma and wound. For the patients who have to wear collecting device for a long term, the most common problems are peristomal skin complications. These complications includes skin excoriation or irritation from faecal output; mechanical irritation related to the removal of adhesives appliances; allergies or sensitivities to chemicals or polymers; folliculitis (infected hair follicles); and monillia/candida albicans which is caused by yeast infection.

Faecal enzymes, moisture and chemical/biological irritants (Berg, 1998; Buckingham and Berg, 1986) are three major contributing factors for peritomal skin complications. Faecal enzymes, in particular; proteases and lipases in stomal output have been reported to be one of the major factors in breaking down skin integrity causing irritation (Buckingham and Berg, 1986). About 45% of patients will suffer from a peristomal skin disorder (Herlufsen et al., 2006). Thus, prevention and management of peristomal skin complications are critical components of ostomy care (Rolstad et al., 2004).

Generally, almost all of skin adhesive agents comprise a polymer or copolymer in which hydrocolloids, such as water absorbing, swollen particles are dispersed. To soften the polymer or copolymer, plasticizers, such as mineral oil, or poly(propylene oxide) have to be added.

U.S. Pat. No. 6,437,038 describes a pressure sensitive adhesive composition comprising 15-60 butyl rubber, 15-60% one or more tackifying resins, 1-10% a tackifying liquid constituent, e.g. paraffin oil, and 20-60% of one or more hydrocolloids.

U.S. Pat. No. 4,551,490 describing adhesives containing SIS/SI, PIB/butyl rubber, tackifier, mineral oil and hydrocolloids.

U.S. Pat. No. 6,933,342 describes a formulation comprising a triblock copolymer, a terpene resin, a liquid and EVA polymer wherein the EVA polymer preferably possess a vinyl acetate content of more than 50% by weight.

The use of hydrocolloid pressure sensitive dressings and an ostomy seal has been described in, for example, U.S. Pat. No. 6,685,683 disclose skin barrier product comprising starch for modifying of the rheological properties of a zone of an adhesive flexible skin barrier product layer for an ostomy appliance.

U.S. Pat. Nos. 3,712,304 and 3,799,166 describes an ostomy seal made from starch and gelatinized starch cross-linked with glyoxal.

U.S. Pat. No. 5,633,010 discloses an invention of adhesive composition comprising a hydrophobic unsaturated aliphatic homopolymer cross-linked by a dose of radiation, a compatible tackifier, and at least one hydrocolloid absorbent. These adhesive compositions exhibit consistently high wet integrity, and thus provide dressings that maintain their form and impart a minimum amount of hydrocolloid residue.

US patent 2011/0251300 describes a soft shapeable adhesive paste. The composition can be used for smoothing out irregular skin area for subsequent secure attachment and has improved erosion resistance, without compromising the moisture absorption, ease of removal or introducing other possible adverse effects.

US patent 2010/0204632 disclosure permeable pressure sensitive adhesive by using polar oils and polar polyethylene copolymers. These adhesives have a very high moisture vapor transmission rate, which make them breathable and very skin friendly.

US patent 2011/0251542 describes polyisobutylene can be used as a tackifying agent which lowers the amount of cell stripping compared to resins.

WO 2009/006901 describes polyisobutylene with low glass transition temperature can be added to the polar adhesives, even though apolar in nature.

US patent 2011/0118363 describes using polar oil or fat including (a) at least one triglyceride, and/or at least one fatty acid of the formula R—CO₂H, wherein R is C3 to C30 alkyl group; and (ii) at least one homopolymer, and/or copolymer. This adhesive composition carries unique features of skin friendliness, e.g. hypoallergenicity, painless removal, repositionability, and ability to initiate natural antimicrobial activities.

None of the adhesives described above contain butters derived from plants and animals, such as shea butter.

Generally, mineral oil is widely used in adhesive formulations; however shea butter which is typically used in cosmetic applications, has not been reported in wound care adhesive or ostomy seal applications. By substituting shea butter for mineral oil, this skin friendly, biocompatible, multifunctional component gives more advantage and provides more and unexpected benefits for skin than biologically inert oils such as mineral oil.

Specifically, Applicants have found that butters derived from plants and animals are useful in reducing the skin irritation experienced by ostomy wearers. Useful butters include, but are not limited to, shea, cocoa, orange peel, aloe and animal butters derived from animal milk such as cow milk. By way of example, shea butter contains many fatty acids: e.g. oleic acid (40-60%), stearic acid (20-50%), linoleic acid (3-11%), palmitic acid (2-9%), linolenic acid(<1%) and arachidic acid(<1%). These carboxylic groups provide an excellent skin adhesive, and at same time will buffer skin surface pH at a natural level of pH 5.5 or below to 7 and thus reduce enzyme activity.

### SUMMARY OF THE INVENTION

The present invention relates to a pressure sensitive adhesive composition comprising:
a. 2 to 45% by weight of a homopolymer or copolymer selected from the group consisting of ethylene vinyl acetate, ethylene vinyl acetate carbon monoxide, ethylene butyl acetate, ethylene vinyl alcohol, ethylene butyl acrylate, ethylene butyl acrylate carbon monoxide, polystyrene copolymers and mixtures thereof;
b. 1 to 10% by weight of a butter selected from the group consisting of shea, avocado, orange peel, aloe or animal derived butter and mixtures thereof;
c. 15-45% by weight of a tackifier selected from the group consisting of natural rosin, modified rosin, glycerol ester of natural rosin, glycerol-ester of modified rosin, pentaerythritol ester of natural rosin, pentaerythritol ester of modified rosin, phenolic modified terpene resin, aliphatic petroleum hydrocarbon resin, cycloaliphatic resin and mixtures thereof.

### BRIEF DESCRIPTION OF THE DRAWINGS

Properties of the invention are shown in more detail in FIGS. 1 and 2, which show the shear modulus G*[Pa] vs. temperature and loss tangent (tan δ) curve vs. the temperature profiles of the compositions according to the invention.

FIG. 1 shows the results of elastic modulus G* and tangent (tan δ) curves for the samples 13-76E, 13-76F and 13-76G from 0° C. to 60° C.

Of all three samples have the same base polymer Kraton 1114 PT, styrenic block copolymer and other ingredients, only slightly amount % difference. Sample 13-76F contains no shea butter, sample 13-76G contains 1.62% and sample 13-76E has 1.81% of total weight. It shows a significant difference of elastic modulus change when the shea butter increases from 0% to 1.81%, G* from 2.56×10⁵ to1.42×10⁵ Pa at 32.4° C., that is, a 44% change G* value in comparison of sample 13-76F with 13-76E. This indicates that even a small amount of shea butter will play a role of softening the adhesive mixture. Sample 13-76G has a similar result with G* decreasing to 34% when the amount of shea butter increases from 0% to 1.62%.

FIG. 2 shows the results of elastic modulus G* and tangent (tan δ) curves for the samples 19-74B and 19-75B from 0° C. to 60° C.

Sample 19-74B and 19-75B base polymer are Elvax 260, polyethylene vinyl acetate block copolymer, the tackfier is Arkon P115. The other ingredients are the same as above three samples. Sample 19-75B has 0.45% more shea butter than sample 19-74B, however, the elastic modulus are changes 45% from 4.1×10⁵ to 2.2×10⁵ Pa at 32.7° C. This also demonstrates that shea butter as rheology modifier and softening agent is a critical material to control the adhesive properties in the designed elastic modulus range.

### DETAILED DESCRIPTION OF THE INVENTION

The present invention relates to a pressure sensitive adhesive composition comprising:
a. 2 to 45% by weight of a homopolymer or copolymer selected from the group consisting of ethylene vinyl acetate, ethylene vinyl acetate carbon monoxide, ethylene butyl acetate, ethylene vinyl alcohol, ethylene butyl acrylate, ethylene butyl acrylate carbon monoxide, polystyrene copolymers and mixtures thereof;
b. 1 to 10% by weight of a butter selected from the group consisting of shea, avocado, orange peel, aloe or animal derived butter and mixtures thereof;
c. 15-45% by weight of a tackifier selected from the group consisting of natural rosin, modified rosin, glycerol ester of natural rosin, glycerol-ester of modified rosin, pentaerythritol ester of natural rosin, pentaerythritol ester of modified rosin, phenolic modified terpene resin, aliphatic petroleum hydrocarbon resin, cycloaliphatic resin and mixtures thereof.

It has been found that the pressure sensitive adhesive compositions with butters derived from plants and animals exhibit unique features that can be used for a variety of applications for medical devices, such as skin adhesives, wound dressings, and ostomy seals. Features of good adhesion to skin, high water absorption, easy shapeable and easy moldable, skin friendliness are obtained by combined with other copolymer such as polyethylene copolymer, poly(isobutylene) and optionally other ingredients, e.g. one or more tackifier resins, and one or more hydrocolloids. For example, Shea butter, a complex mixture of fat and vitamins A and E, on the other hand, will help moisturize skin, protect skin from damage, and in some case even slow down or cure skin diseases. Shea butter also plays a role as rheology modifier. These multiple functions lead to a very special character of skin friendliness, e.g. protection, prevention, healing, prepositional and easy of removal of pressure sensitive adhesives for skin fixation, wound dressing and ostomy seal applications.

By introducing shea butter, the stability issues due to phase separation or migration are avoided. The properties of shea butter as an amphiphile allow it to combine polar and non-polar materials. Dynamic mechanical analysis shows uniform curves of (G*) and tan (δ) with combined rheological expressions of both polar and non-polar parts.

By addition of shea butter, a rheology modifier and plasticizer for ostomy and other medical devices, it is possible to achieve a softer paste that is easier to shape. Shea butter in ostomy paste and medical device fixation applications has not been reported. Shea butter will buffer the skin in the proper pH range and reduce enzyme activity, to prevent both skin diseases to happen and the paste to break down either. Other suitable butters include, but are not limited to, avocado, orange peel, aloe or animal derived butter and mixtures thereof. Said butters comprise 1-10% of the final formulation.

The pressure sensitive adhesive composition of the present invention differs from the compositions disclosed in U.S. Pat.Nos. 2011/0251542A1 and 2011/0251300A1 in that they do not include shea butter.

The pressure sensitive adhesive composition of the present invention differs from the composition of disclosed from in U.S. Pat. Nos. 2010/0204632A1 in that they comprise the polyethylene copolymer has a melt flow index below 2 g/10 min and in that the polyethylene copolymer has a melt flow index above 2 g/10 min.

It is also found that shea butter as softening agent and moisturizer leads to a comfortable texture and feel on the part of the user.

In this present invention, the design approach is directed toward obtaining an adhesive to be moldable, easily shaped, moisture absorbing skin barrier, at same time also can be stretched, compressed or molded to fit the exact shape and size as required, which means adhesive should not have memorable property in order to keep the designed shape. Thus the G′ has to keep certain high level while still keep the properties such as tacky and moldable. To achieve the above required properties, adhesive G′ and tan δ in designed range are very critical. By addition of shea butter to the adhesive composition yielded surprisingly desired results. By adjusting the amount of shea butter, both the rheology G′ and tan delta can be obtained in the designed range.

In one embodiment of the present invention, the adhesive composition comprises shea butter with vitamins E and A and at least one homopolymer, and/or block copolymer. The homopolymer includes, but is not limited to isobutylene, polyethylene, polypropylene, polybutylene, polystyrene.

A suitable block copolymer contains at least two monomers which have very different glass transition temperatures so that they are immiscible in each other and phase separated at room temperature. Examples of such copolymers include, but are not limited to, styrene isoprene styrene, styrene butadiene styrene, styrene propylene styrene, and ethylene vinyl acetate and ethylene acrylate.

The primary polymers used in the adhesive composition are ethylene copolymers, the polymer should contain considerable amount of a polar component to soften and get water permeability. Preferably, the ethylene parts of the copolymer can form crystalline areas that ensure the cohesive strength of the adhesive. Said primary polymers comprise 2-45% of the final formulation.

In one embodiment of the invention, the polar polyethylene copolymer is selected from the group consisting of ethylene vinyl acetate, ethylene vinyl acetate carbon monoxide, ethylene butyl acetate, ethylene vinyl alcohol, ethylene butyl acrylate, ethylene butyl acrylate carbon monoxide and combinations thereof.

The polar polyethylene copolymer is preferably ethylene vinyl acetate with 18-30% weight of vinyl acetate; especially prefer 28% of vinyl acetate.

In one of embodiment, the present invention, the adhesive composition comprises shea butter with vitamin E and A and at least one homopolymer, and/or copolymer, and a tackifier.

The tackifiers applicable to this invention adhesive include, but not limited to, natural rosin, modified rosin, glycerol ester of natural rosin, glycerol-ester of modified rosin, pentaerythritol ester of natural rosin, pentaerythritol ester of modified rosin, phenolic modified terpene resin, aliphatic petroleum hydrocarbon resin, and cycloaliphatic resin.

Tackifier can used to control the tack of the adhesive and reduce moduli and increase glass transition temperature.

Said tackifiers comprise 15-45% of the final formulation, more preferably from 20-35%.

In one of embodiment, the present invention, the adhesive composition comprising plasticizer selected from the group of mineral oil, citrate oil, paraffin oil, phthalic acid esters, adipic acid esters and liquid or solid resin.

In one embodiment of the present invention, the adhesive composition further comprises a polyethylene wax.

In another embodiment of the invention, the composition of the ostomy seals or skin fixation device further comprises other ingredients selected from a group of antioxidants, stabilizers, fillers, pigments, flow modifiers, and active ingredients such as drugs.

In one of embodiment, the present invention the adhesive composition comprises shea butter with vitamin E and A, and at least one homopolymer, and/or copolymer, a tackifier and a hydrophilic liquid absorber or gel-thickener.

Suitable hydrophilic liquid absorbers or gel-thickeners are selected from naturally occurring hydrocolloids, semisynthetic hydrocolloids and synthetic hydrocolloids.

More particularly, the hydrocolloids are preferably selected from guar gum, locust bean gum, pectin, alginates, gelatin, xanthan and/or gum karaya; cellulose derivatives such as sodium carboxymethylcellulose, methylcellulose and hydroxypropylmethylcellulose and/or sodium starch glycolate and/or polyvinyl alcohol and/or polyethylene glycol.

It is preferred to use a combination of two or more hydrocolloids. It is especially preferred to use combination of guar gum and sodium carboxymethylcellulose as the hydrocolloid component.

In one embodiment of the invention, the content of hydrocolloid is 20-60% of the total composition, more preferably from 30-45%.

According to one embodiment of the invention, the final paste has an elastic modulus of less than 1000000 Pa, preferable less than 300000 Pa at 1 Hz (1% deformation, 32° C.).

According to one of the inventions, the final paste has a tan (δ) above 0.8, preferably above 1.0 at 1 Hz (1% deformation, 32° C.).

The following non limiting examples illustrate the practice of the present invention.

### EXPERIMENTAL

Materials and methods

### Materials

TABLE 1

| Description | Manufacturer | Property |
|---|---|---|
| Oppanol 10 | BASF | Base polymer |
| Elvaloy ® AC 1125 | Dupont | Elastomer |
| Liposorb TS | Lipo Chemical | Rheology |
| | | modifier |
| Wingtack 95 Flake | Eastman | Tackifier |
| Guar NT 3500F | TIC Gums Inc | Water absorbent |
| Carboxymethylcellulose F1- | Amtex | Water absorbent |
| 4000XX | | |
| Microcrystalline Cellulose | FMC Bio polymer | Filler |
| (MCC) | | |
| LipoButter, Refined Shea | Lipo Chemical | Rheology |
| (non-organic) | | modifier |

PIB: Polyisobutylene available under trademark oppanol from BASF chemical company
Poly ethylene vinyl acetate and poly ethylene acrylate from Dupont chemical company.
Kraton D1114PT: Styrene-isoprene-styrene copolymer from Kraton company.
Wingtack 95: hydrocarbon resins from Cray valley.
Amtex CMC: Sodium carboxymethylcellulose from Amtex chemicals
GuarNT3500F powder: from TIC Gums, Inc.
MCC: Microcrystalline Cellulose from FMC Bio polymer with trade name PH-200NF.
Lipobutter Refined Shea: Refined shea butter from Lipo chemicals.
Liposorb® TS: Sorbitan tristearate from Lipo chemicals.

Elvax 260, Elvaloy and Kraton polymer provides the cohesive strength. PIB provides the main foundation of the moldable adhesive. Shea butter and Liposorb TS serves as rheology modifiers and skin friendly moisturizing agents. MCC plays the role as filler. Amtex CMC and guar gum absorb fluids. Wingtack 95 helps adhesive and initial tack property.

### Example 1-2

Preparation of adhesive materials according to the invention having the composition stated in Table 2 below:

TABLE 2

| | Mix EL1108161 | Mix EL1108241 |
|---|---|---|
| Description | % | % |
| Elvaloy ® AC1125 | 5.44 | 5.43 |
| LipoButter, Refined Shea | 2.44 | 2.43 |
| Oppanol 10 | 33.52 | 33.49 |
| Microcrystalline Cellulose | 5.23 | 5.30 |
| (MCC) | | |
| Wingtack 95 Flake | 17.38 | 17.35 |
| Amtex CMC F1-4000XX | 22.12 | 22.70 |
| Liposorb TS | 6.28 | 6.32 |
| Guar NT 3500F | 7.61 | 6.97 |
| Total | 100.00 | 100.00 |

### Method of Mixing

Elvaloy® AC 1125 was added in to the Z-mixer at 165° C. and kneaded by the mixing machine for 15 minutes. Shea butter was added and mixing was continued at 165° C. for 15-30 minutes, after which Oppanol 10 was added until the blend was homogenous. Then MCC was added until there were no polymer chunks and whole mixture was homogenous. To the mixture Wingtack 95 was added and kneaded for 15 min, and then following Amtex CMC, Liposorb TS were added in order at intervals of a few minutes without continuing to heat the mixer. When the temperature cooled to 100° C. or below, Guar gum was added until a homogeneous mixture was formed.

### Examples 3-5

Following similar procedure disclosed in the Example 1-2 adhesive composition according to the invention having the compositions stated in Table 3 were prepared.

TABLE 3

| | Mix | Mix | Mix |
|---|---|---|---|
| | 13-76E | 13-76F | 13-76G |
| Component | % | % | % |
| Kraton1114PT | 4.02 | 4.16 | 4.25 |
| Oppanol 10 | 28.79 | 31.16 | 31.01 |
| Wingtack95 | 17.79 | 18.19 | 17.68 |
| Amtex CMC | 23.84 | 25.85 | 25.94 |
| Guar NT | 8.83 | 7.28 | 7.26 |
| 3500F | | | |
| MCC | 4.72 | 5.22 | 5.02 |
| Shea butter | 1.81 | 0.00 | 1.62 |
| Liposorb TS | 6.18 | 8.14 | 7.21 |
| Total | 100.00 | 100.00 | 100.00 |

The examples in Table 3 comprise mixtures different from examples 1-2. Kraton 1114PT, Styrenic block copolymer, replaced Elvaloy® AC 1125 as cohesive strength material.

### Examples 6-7

Following similar procedure disclosed in the Example 1-2 adhesive composition according to the invention having the compositions stated in Table 4 were prepared.

TABLE 4

| | Mix 19-74B | Mix 19-75B |
|---|---|---|
| Component | % | % |
| Elvax260 | 8.34 | 8.33 |
| Oppanol 10 | 29.41 | 29.30 |
| Arkon p115 | 17.90 | 18.03 |
| MCC | 5.56 | 5.54 |
| Amtex CMC | 22.60 | 22.53 |
| Shea butter | 3.66 | 4.11 |
| Liposorb TS | 5.57 | 5.21 |
| Guar gum | 6.96 | 8.94 |
| | 100.00 | 100.00 |

The Table 4 the samples differ from examples 1-2 in that Elvax 260, polyethylene vinyl acetate block copolymer, replaced Elvaloy® AC 1125 as cohesive strength material.

### Examples 8-9

### Water Absorption Measurement

### Sample Prepare Method

The adhesive was pressed between two pieces of silicone paper and PU-film paper by compressor mold at 85° C. and 10000 lb force with thickness of 1 mm, the sample covered with the release liner with thickness 0.18 mm and PU-film with thickness 0.02 mm. A sample of 1′ diameter was then punched out while the double sticky tape was adhering on the PU-film.

### Method of Determination of Water Absorption

### Water Absorption Measurement

The sample with PU-film and double sticky film was weighed and placed in container with 0.9% saline at 37° C. oven. After 2hours, 4 hours, 6 hours, 18 hours and 24 hours, the sample was removed from the container, the water was shaken off, and the sample was weighed again. The increase of weight was recorded as the water absorption.

The water absorption was calculated as

Water absorption after 2 hours=M(2 hours)−M(start)/5.026[g/cm²]

TABLE 5
Water Absorption (g/10 cm²)

| Sample # | 2 hrs | 4 hrs | 6 hrs | 18 hrs | 24 hrs |
|---|---|---|---|---|---|
| EL1108161 | 2.84 | 4.16 | 5.15 | 9.56 | 9.39 |
| EL1108241 | 3.17 | 4.46 | 5.09 | 10.18 | 8.62 |

Samples EL1108161 and EL1108241 showed a similar level of water absorption at 2 h, 4 h, 6 h 18 h and 24 h. It shows that a small percentage of change of shea butter in the adhesives composition did not play the big role in water absorbance as rheology relatively. Water absorbance reaches the peak at 18 hour for both of the samples.

### Examples 10-12

TABLE 6
Water Absorption (g/10 cm²)

| Sample # | 2 hrs | 4 hrs | 24 hrs |
|---|---|---|---|
| 13-76E | 3.63 | 4.75 | 10.09 |
| 13-76F | 3.27 | 4.57 | 9.62 |
| 13-76G | 3.63 | 4.61 | 8.58 |

Samples 13-73E, 13-76F and 13-76G also showed the similar level of water absorption at 2 h, 4 h, 6 h 18 h and 24 h. It gives the same conclusion even with different the base polymer that small amount of change of shea butter in the adhesives composition has not significant affect in water absorbance as rheology relatively.

### Example 13-14

TABLE 7
Water Absorption (g/10 cm²)

| Sample # | 2 hrs | 4 hrs | 6 hrs | 18 hrs | 24 hrs |
|---|---|---|---|---|---|
| 19-74B | 2.40 | 4.39 | 5.71 | 11.66 | 12.86 |
| 19-75B | 2.46 | 4.07 | 5.42 | 11.71 | 13.21 |

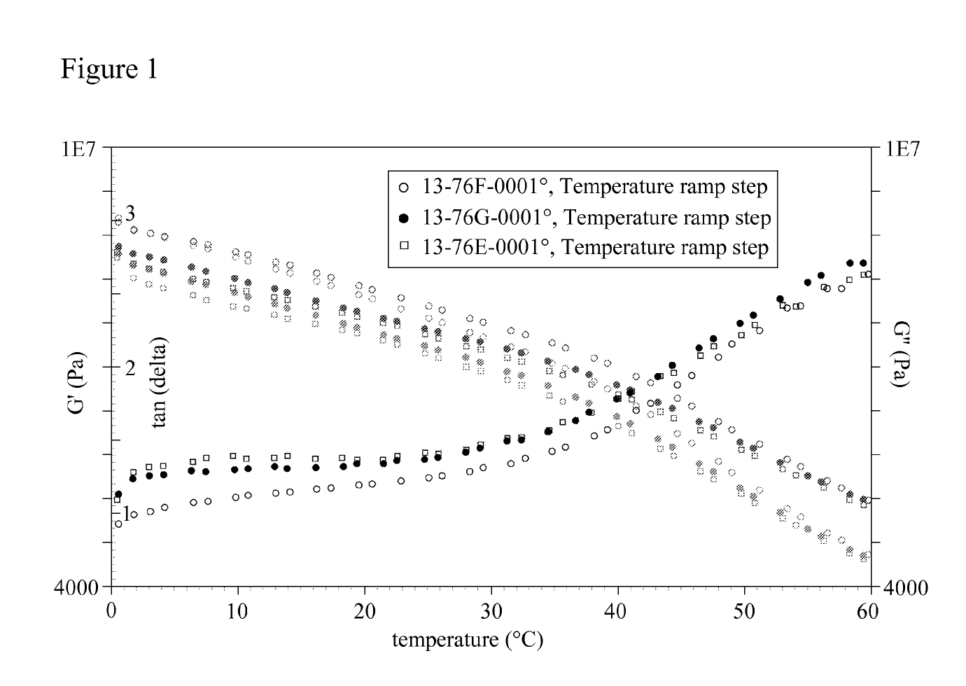

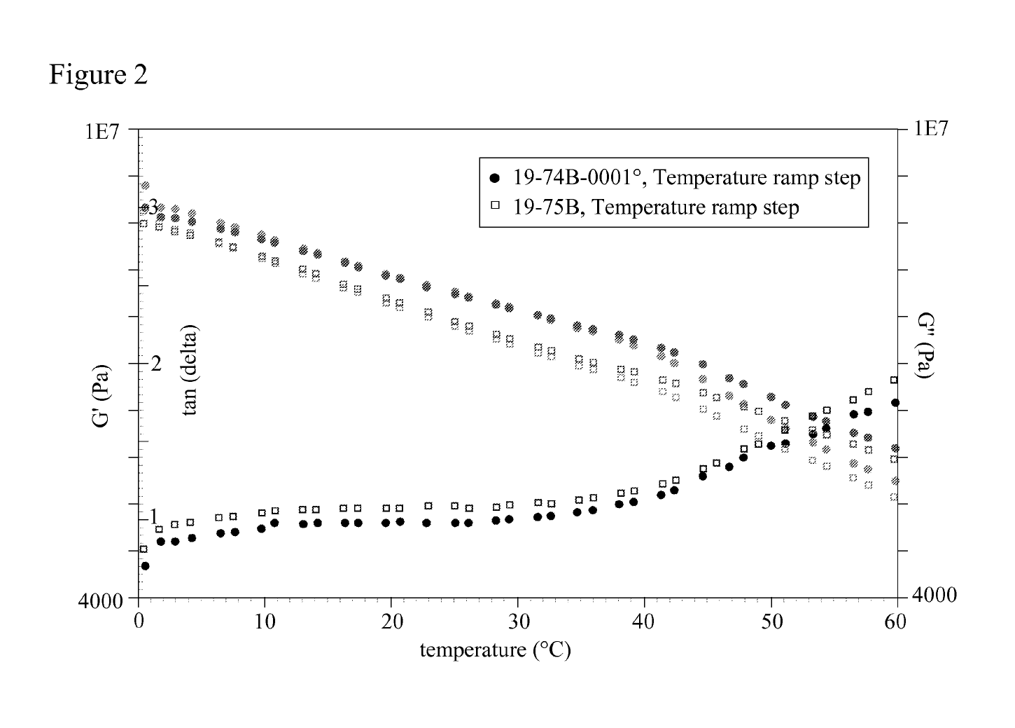

Samples 19-74B and 19-75B have a higher percentage of base polymers than examples in Tables 5 and 6. Thus the initial water absorbance is lower than in the polymers in Tables 5 and 6, however, the water absorbance keeps increasing even after 18 hour, and is highest at 24 hours of all above samples. The small percentage increase in shea butter in the adhesive composition has no significant effect on water absorbance, although it has a large effect on the elastic modulus (see FIGS. 1 and 2). Moreover, the cohesive strength is maintained due to the high percentage of the homopolymer of copolymer.

### Determination of Probe Tack

Probe tack test were carried out with probe tack instrument of EZlab

Compatible from ChemInstruments;

The samples were prepared in the same way as water absorbance except with double sticky tape on one side.

The test results show a little slightly higher probe tack than for the existing commercial product available under the trademark Eakin cohesive seal.

### Example 15

TABLE 8

| | Sample # | | |
|---|---|---|---|
| | 13-76E | 13-76F | 13-76G |
| Probe | 199 | 126 | 198 |
| Tack (g) | | | |
| Shea butter, | 1.81 | 0 | 1.62 |
| % | | | |

The example 15 illustrates that a small amount of shea butter increases the probe tack.

Example 16

TABLE 9

Sample #

| | EL1108161 | EL1108241 |
|---|---|---|
| Probe | 182 | 188 |
| Tack (g) | | |
| Shea butter, | 2.44 | 2.43 |
| % | | |

Example 16 shows the probe tack of two formulations with shea butter.

### Example 17

TABLE 10

Sample #

| | 19-74B | 19-75B |
|---|---|---|
| Probe | 65 | 97 |
| Tack (g) | | |
| Shea butter, | 3.66 | 4.11 |
| % | | |

The results of Table 10 show that a small increase in shea butter concentration raises the probe tack slightly. At same time, the base polymer percentage in the formulation causes the changes of both mechanical and physical properties. Probe tack and water absorbance decrease as long polymer concentration increases.

### Example 18

### Test of Rheological Properties

Test of the elastic properties as measured by Dynamic Mechanical Analysis of composition of examples 13-76E, 13-76 F, and 13-76 in FIG. 1, 19-74B and 19-785B in

FIG. 2. of the present application have been carried out.

### Dynamic Mechanic Analysis (DMA)

The analysis was carried out by using a TA2000 EX apparatus.

Total viscoelastic modulus (*G) and tan δ were determined at a frequency sweep 1% from temperature 0° C. to 60° C.

The three samples 13-76E, 13-76 F and 13-76 had similar formulations except sample 13-76F without shea butter. The samples 19-74B and 19-78B base polymer are polyethylene vinyl acetate copolymer, while the other above three samples base polymer contain Kraton 1114PT, a styrene-isoprene-styrene copolymer.

1. A pressure sensitive adhesive composition comprising:
a. 2 to 45% by weight of a homopolymer or copolymer selected from the group consisting of polyisobutylene, ethylene vinyl acetate, ethylene vinyl acetate carbon monoxide, ethylene butyl acetate, ethylene vinyl alcohol, ethylene butyl acrylate, ethylene butyl acrylate carbon monoxide, polystyrene copolymers and mixtures thereof;
b. 1 to 10% by weight of a butter selected from the group consisting of shea, avocado, orange peel, aloe or animal derived butter and mixtures thereof; and
c. 15- 45% by weight of a tackifier selected from the group consisting of natural rosin, modified rosin, glycerol ester of natural rosin, glycerol-ester of modified rosin, pentaerythritol ester of natural rosin, pentaerythritol ester of modified rosin, phenolic modified terpene resin, aliphatic petroleum hydrocarbon resin, cycloaliphatic resin and mixtures thereof.

2. A pressure sensitive adhesive composition according to claim 1 wherein said butter is shea butter.
